(19) United States
(12) Patent Application Publication
(10) Pub. No.: US 2016/0183806 A1
(43) Pub. Date: Jun. 30, 2016

(54) PHOTOACOUSTIC APPARATUS

(71) Applicant: CANON KABUSHIKI KAISHA, Tokyo (JP)

(72) Inventor: Hiroshi Yamamoto, Kawasaki-shi (JP)

(21) Appl. No.: 14/975,010

(22) Filed: Dec. 18, 2015

(30) Foreign Application Priority Data
Dec. 25, 2014 (JP) PCT/JP2014/084219

(51) Int. Cl. A61B 5/00

(52) U.S. Cl. CPC A61B 5/0095

(57) ABSTRACT

A photoacoustic apparatus that improves absorption coefficient measurement accuracy by obtaining information on a subject after performing correction in accordance with amounts of light emitted to the subject includes a photoacoustic wave detection unit that detects photoacoustic waves generated from a subject when a light source irradiates the subject with light and output detection signals and a signal processing unit that performs signal processing to obtain information on the subject in accordance with the detection signals. The signal processing unit obtains information on the subject after performing correction in accordance with amounts of light emitted to the subject.

### TECHNICAL FIELD

Aspects of the present invention generally relate to a photoacoustic apparatus.

### BACKGROUND ART

As one of methods for obtaining optical characteristic values, such as an absorption coefficient, in a subject, photoacoustic tomography (PAT) utilizing ultrasonic waves has been proposed. Apparatuses utilizing the PAT (hereinafter referred to as “photoacoustic apparatuses”) at least include a light source and probes.

First, when the light source irradiates a living body with pulsed light, the light is dispersed and propagated in the object. A light absorber included in the subject absorbs the propagated light and generates photoacoustic waves (typically, ultrasonic waves). The photoacoustic waves are received by the probes and output as detection signals and the detection signals are analyzed so that an initial sound pressure distribution caused by presence of the light absorber included in the subject may be obtained. According to NPL 1, a sound pressure P of an ultrasonic wave obtained due to the light absorption from the light absorber included in the subject in the PAT is represented by the following expression.

P=Γ·μa·Φ (1)

In Expression (1), “P” denotes an initial sound pressure. “Γ” denotes a Grueneisen coefficient and is obtained by dividing a product of a volume expansion coefficient β and a square of a sound speed c by a specific heat Cp. “μa” denotes an absorption coefficient of the light absorber and “Φ” denotes an amount of light absorbed by the light absorber. According to this expression, an absorption coefficient relative to an initial sound pressure in an arbitrary position may be obtained by taking an amount of light which reaches the position into consideration. Since different light absorbers have different absorption coefficients, a distribution of the light absorber included in the subject, such as a distribution of blood vessels, may be obtained by obtaining a distribution of absorption coefficients of the subject.

In a case where the subject is a breast, a pressure applied to the breast held by a curved holding section is smaller than a pressure applied to the breast held by a flat holding section, and therefore, a burden of an examinee is smaller when the pressure is applied to the breast held by the curved holding section. Accordingly, in PTL 1, a breast is held by a bowl-shaped holding section and a light irradiation section and probes integrally scan the holding section so that photoacoustic waves are detected from the breast.

### CITATION LIST

### Patent Literature

PTL 1: Japanese Patent Laid-Open No. 2012-179348

### Non Patent Literature

NPL 1: M. Xu, L. Wang “Photoacoustic imaging in biomedicine, Review of scientific instruments, 77(4), 041101-1-041101-22(2006)

However, in a case of an apparatus of PTL 1, a distance between the light irradiation section and the holding section varies depending on a scanning coordinate of the light irradiation section. In general, an acoustic matching section is disposed between the light irradiation section and the holding section so as to perform acoustic matching. In a case where water is used as the acoustic matching section 11 as described in PTL 1, when light having a wavelength of 750 nm, for example, propagates in the water, intensity of the light is attenuated at a rate of approximately 2.6%/cm. In a case where a distance from the light irradiation section to a center portion of the holding section and a distance from the light irradiation section to a periphery portion of the holding section are different from each other by 5 cm, amounts of light emitted to the subject are different by approximately 12%. As represented by Expression (1) above, an initial sound pressure distribution in the inside of the subject is proportional to an amount of light emitted to the subject. Therefore, if amounts of light emitted to the subject are seen to be equal to one another irrespective of a scanning coordinate of the light irradiation section, accuracy of measurement of an absorption coefficient is degraded.

### SUMMARY

According to an aspect of the present invention, a photoacoustic apparatus includes a photoacoustic wave detection unit configured to detect photoacoustic waves generated from a subject when a light source irradiates the subject with light and output detection signals, and a signal processing unit configured to perform signal processing to obtain information on the subject in accordance with the detection signals. The signal processing unit obtains information on the subject after performing correction in accordance with amounts of light emitted to the subject.

Further features of the present disclosure will become apparent from the following description of exemplary embodiments with reference to the attached drawings.

### BRIEF DESCRIPTION OF DRAWINGS

FIG. 1A is a diagram illustrating a photoacoustic apparatus according to a first embodiment.

FIG. 1B is a diagram illustrating the photoacoustic apparatus according to the first embodiment.

FIG. 1C is a diagram illustrating a correction table stored in a storage unit according to the first embodiment.

FIG. 1D is a flowchart illustrating measurement according to the first embodiment.

FIG. 2 is a diagram illustrating a photoacoustic apparatus according to a second embodiment.

FIG. 3A is a diagram illustrating a photoacoustic apparatus according to a third embodiment.

FIG. 3B is a diagram illustrating a flowchart illustrating measurement according to the third embodiment.

FIG. 4 is a diagram illustrating a photoacoustic apparatus according to a fourth embodiment.

FIG. 5A is a diagram illustrating a photoacoustic apparatus according to a fifth embodiment.

FIG. 5B is a flowchart illustrating measurement according to the fifth embodiment.

FIG. 6A is a diagram illustrating a photoacoustic apparatus according to a sixth embodiment.

FIG. 6B is a diagram illustrating a correction table stored in a storage unit according to the sixth embodiment.

FIG. 6C is a flowchart illustrating measurement according to the sixth embodiment.

FIG. 6D is a flowchart illustrating measurement according to the sixth embodiment.

FIG. 7A is a diagram illustrating a photoacoustic apparatus according to a seventh embodiment.

FIG. 7B is a flowchart illustrating measurement according to the seventh embodiment.

### DESCRIPTION OF EMBODIMENTS

A photoacoustic apparatus according to exemplary embodiments will be described hereinafter.

The photoacoustic apparatus according to the exemplary embodiments includes an acoustic wave detection unit which detects photoacoustic waves generated from a subject when a light source irradiates the subject with light and which outputs detection signals and a signal processing unit which performs signal processing so as to obtain information on the subject in accordance with the detection signals. The signal processing unit obtains the information on the subject after performing correction in accordance with amounts of light emitted to the subject. Specifically, the signal processing unit does not employ a constant value, which is constant irrespective of a light irradiation position, for amounts of light emitted to the subject but performs correction in accordance with differences among light irradiation positions of the light emitted to the subject and differences among distances (light path lengths) in which light emitted from the light source to the subject (or the holding section described below) passes in the acoustic matching section (described below). Since the correction is performed, reliable values of light amounts may be obtained and accuracy of measurement of absorption coefficients may be improved. Since reliable values of light amount distributions are obtained, accuracy of an absorption coefficient distribution may be improved.

Furthermore, the photoacoustic apparatus of the embodiment may include a storage unit which stores a correction table used to correct differences among amounts of light emitted to the subject caused by differences among light irradiation positions of the light emitted to the subject. The signal processing unit obtains the information on the subject after correcting light amounts in accordance with the correction table. Furthermore, the photoacoustic apparatus of the embodiment may include a distance measurement unit which measures distances in which light which is emitted from the light source to the subject passes in the acoustic wave matching unit. The signal processing unit may obtain the information on the subject after correcting the differences among amounts of light emitted to the subject caused by differences among distances measured by the distance measurement unit. If the photoacoustic apparatus includes a storage unit as described above, measurement of distances is not required, and therefore, it is advantageous in that a configuration of the apparatus is simplified. However, if the photoacoustic apparatus includes a distance measurement unit as described above, measurement of distances is actually performed, and therefore, light amounts may be more reliably corrected.

Furthermore, the photoacoustic apparatus of the embodiment may include a position control unit which controls relative positions of the light source and the subject. The position control unit may control at least one of a position of the light source and a position of the subject. An amount of light emitted to the subject varies depending on a difference of positions obtained by the position control unit. Accordingly, the information on the subject may be obtained after correction is performed in accordance with differences among positions of the light source controlled by the position control unit. Note that the storage unit may include a correction table used to correct differences among amounts of light emitted to the subject caused by the differences among the positions obtained by the position control unit, and the information on the subject may be obtained after correction is performed using the correction table.

Note that a target of the correction performed by the signal processing unit is a parameter used to calculate the absorption coefficient μa in Expression (1) and any parameter may be employed as long as the parameter changes depending on the differences among the amounts of light emitted to the subject. Although the parameter is typically Φ (an amount of light absorbed by the light absorber) in Expression (1), other parameters used to calculate Φ may be used. Furthermore, a value of an amount of light measured by a light-amount measurement unit in an embodiment described below may be used as the light amount Φ of the light emitted to the subject. Furthermore, a value calculated using a value of a light amount calculated in accordance with an output value of the light of the light source, that is, an output value of the light of the light source and transmissivity of a medium (such as a light transmission path) through which the light emitted from the light source passes before reaching the subject may be used as the light amount Φ.

As a representative example, the signal processing unit of the embodiment further includes a storage unit which stores a correction table having correction coefficients associated with individual light irradiation positions of light emitted to the subject. In this case, the signal processing unit calculates the light amounts Φ by correcting values of amounts of light emitted to the subject in accordance with the correction coefficients included in the correction table and calculates information such as absorption coefficients) of the subject from the light amounts Φ. Note that the calculation of the absorption coefficients in this embodiment means calculation of distributions of absorption coefficients (absorption coefficients and positional information of the absorption coefficients), and the same is true on the description below.

The photoacoustic apparatus of the exemplary embodiments will be described in detail hereinafter using concrete examples.

### First Embodiment

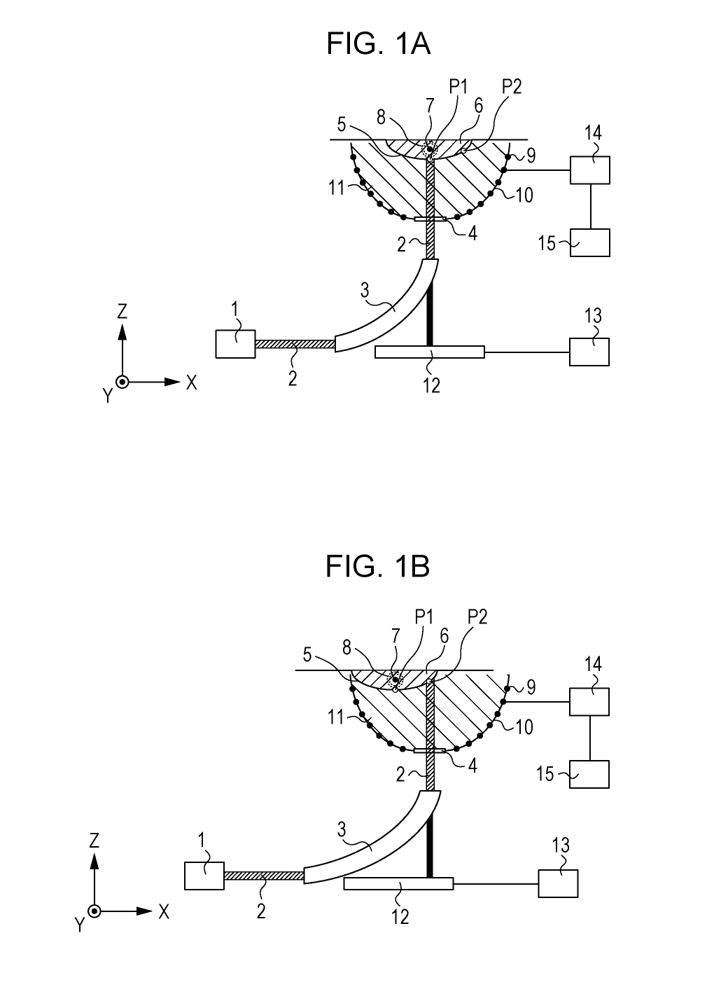

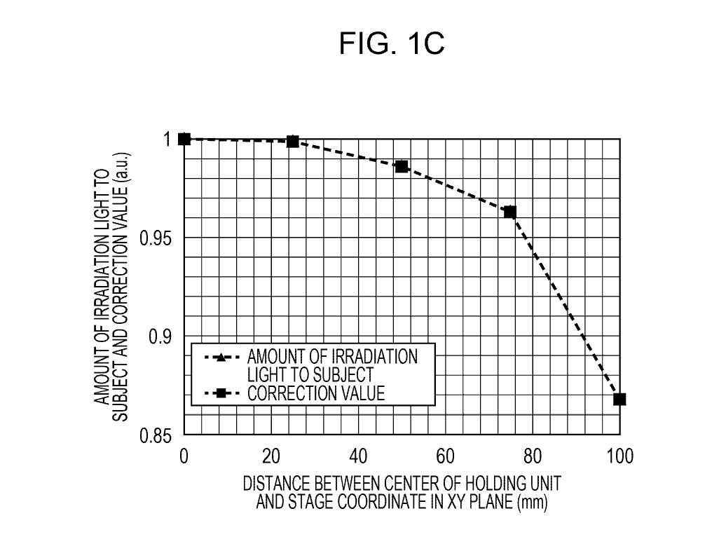

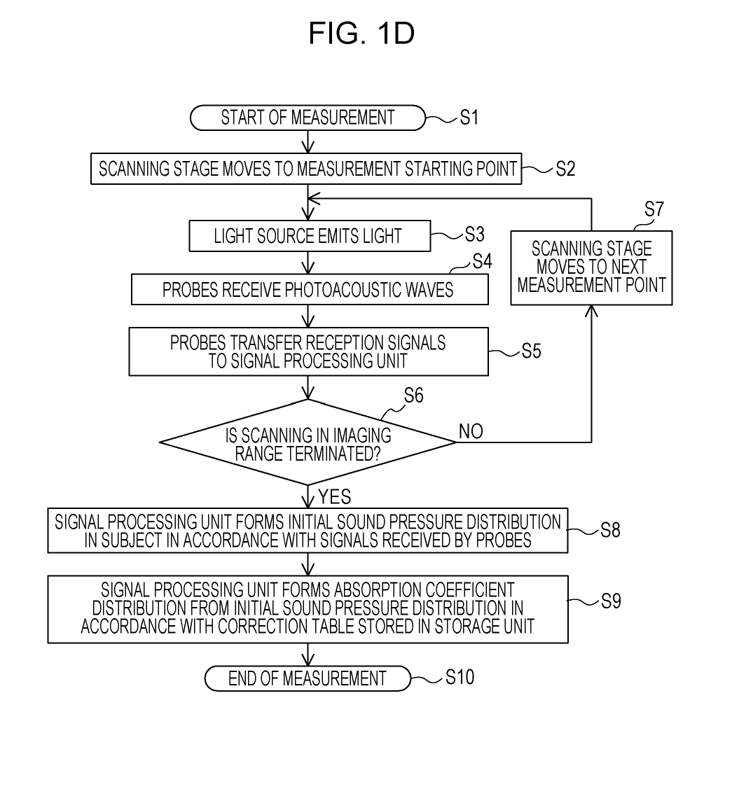

A configuration of this embodiment will be described with reference to FIG. 1. In FIGS. 1A and 1E, a reference numeral 1 denotes a light source, 2 denotes light, 3 denotes an optical waveguide, 4 denotes a light irradiation section, 5 denotes a holding section, 6 denotes a subject, 7 denotes a light absorber, 8 denotes photoacoustic waves, 9 denotes acoustic wave detection units (probes), 10 denotes a probe supporting section, 11 denotes an acoustic matching section, 12 denotes a scanning stage, 13 denotes a stage control unit, 14 denotes a signal processing unit, and 15 denotes a storage unit. FIG. 1A is a diagram illustrating a state in which the light irradiation section 4 and the probe supporting section 10 scan a center portion P1 of the holding section 5. FIG. 1B is a diagram illustrating a state in which the light irradiation section 4 and a center of the probe supporting section 10 scan a periphery portion P2 of the holding section 5. FIG. 1C is a diagram illustrating a correction table stored in the storage unit. FIG. 1D is a flowchart illustrating measurement according to this embodiment.

In this embodiment, the light source 1 is a titanium-sapphire laser. The titanium-sapphire laser has a wavelength of 797 nm, an output of 120 mJ, a frequency of 20 Hz, and a pulse width of 10 nanoseconds, for example. The light 2 emitted from the light source 1 is transmitted through the optical waveguide 3 which is a bundle fiber including a bundle of a plurality of optical fibers. The light 2 output from the optical waveguide 3 passes through the light irradiation section 4 and is incident on the subject 6 through the holding section 5. Here, the light irradiation section 4 is a plate of polycarbonate. The center portion P1 of the holding section 5 is separated from the light irradiation section 4 in a Z direction by 100 mm, and the center portion P1 is separated from the periphery portion P2 of the holding section 5 in the Z direction by 50 mm. The light dispersed in the subject 6 is absorbed by the light absorber 7. Then the photoacoustic waves 8 generated from the light absorber 7 propagate through the subject 6, the holding section 5, and the acoustic matching section 11 and are received by the probes 9. Note that the acoustic matching section 11 is water. Furthermore, the probes 9 are capacitive micromachined ultrasonic transducers (CMUTs). The probes 9 are arranged on the probe supporting section 10 of a cup shape such that directions of the highest sensitivities of reception directivities of at least some of the probes 9 intersect with one another. The optical waveguide 3 and the probe supporting section 10 perform scanning using the scanning stage 12, and a pattern of the scanning is controlled by the stage control unit 13. In this embodiment, the stage control unit 13 causes the optical waveguide 3 and the probe supporting section 10 to perform scanning in an XY plane in a spiral manner. The signal processing unit 14 forms an initial sound pressure distribution in the inside of the subject 6 using the signals received by the probes 9. The signal processing unit 14 further forms an absorption coefficient distribution using the initial sound pressure distribution in accordance with the correction table stored in the storage unit 15. Note that the signal processing unit 14 has light amount data of light emitted to the subject 6 in at least one stage coordinate as a reference.

Next, the correction table stored in the storage unit 15 and a method for using the correction table employed in the signal processing unit 14 will be described with reference to FIG. 1C. An axis of abscissa of FIG. 1C denotes a distance from the center portion P1 of the holding section 5 to a center of the probe supporting section 10 in the XY plane. When a distance from the center portion P1 of the holding section 5 to a stage coordinate in the XY plane is increased, a thickness of the acoustic matching section 11 is increased, that is, a distance (a light path length) in which the light emitted from the light source passes the acoustic matching section 11 is increased. Accordingly, a light attenuation amount in the acoustic matching section 11 is increased. Consequently, an amount of light emitted to the subject 6 is reduced. Therefore, if the absorption coefficient distribution in the subject 6 is formed after it is determined that amounts of light emitted to the subject 6 are equal to one another irrespective of the stage coordinates, an error occurs. Therefore, the storage unit 15 stores correction values for individual stage coordinates, that is, the correction table, such that a correction value of a light amount is reduced as the distance from the center portion P1 of the holding section 5 to a stage coordinate in the XY plane is increased so that an amount of light emitted to the subject 6 is appropriately estimated. The signal processing unit 14 may reduce influence of the differences among amounts of light emitted to the subject in individual stage coordinates by multiplying correction values in the correction table on amounts of light measured by the center portion P1 of the holding section 5 in advance or amounts of light obtained by multiplying transmissivities of components in the light transmission path on outputs of the light source 1.

Next, a flow of the measurement in this embodiment is described with reference to FIG. 1D. First, an operator starts measurement (S1). Then the scanning stage 12 moves to a measurement starting point (S2). The light source 1 irradiates the subject 6 with the light 2 (S3). The probes 9 receive the photoacoustic waves 8 from the subject 6 (S4). Signals received by the probes 9 are transferred to the signal processing unit 14 (S5). A system determines whether imaging in a predetermined range has been terminated (S6). When the system determines that the imaging in the predetermined range has not been terminated, the scanning stage moves to a next measurement point (S7) and the process returns to step S3. When the system determines that the imaging in the predetermined range has been terminated in step S6, the signal processing unit 14 forms an initial sound pressure distribution in the subject 6 in accordance with the signals received by the probes 9 (S8). Subsequently, the signal processing unit 11 forms an absorption coefficient distribution from the initial sound pressure distribution in accordance with the correction table stored in the storage unit 15 (S9). Then the measurement is terminated (S10).

Since the correction table used to appropriately estimate differences among amounts of light emitted to the subject obtained in individual stage coordinates is stored in the storage unit and the signal processing unit refers to the correction table stored in the storage unit when forming information on the inside of the subject as described above, the absorption coefficient distribution may be reliably measured even if different light, attenuation amounts of the acoustic matching section are obtained depending on the stage coordinates. Note that it is not necessarily the case that different correction values in the correction table are used for different stage coordinates. In a case where amounts of light emitted to the subject are seen to be substantially the same in different stage coordinates, for example, the same correction value may be used.

Photoacoustic Apparatus

The photoacoustic apparatus of this embodiment obtains information on the inside of the subject. The photoacoustic apparatus of this embodiment includes, as a basic hardware configuration, the light source, the optical waveguide, the light irradiation section the holding section which holds the subject, the probes which receive photoacoustic waves generated in the subject, the probe supporting section which supports the probes, the acoustic matching section which is used to acoustically connect the holding section and the probes to each other, the scanning stage which causes the optical waveguide and the probe supporting section to scan the holding section, the stage control unit which controls a coordinate of the scanning stage, the signal processing unit which forms information on the inside of the subject using the signals received by the probes, and the storage unit which stores correction values used to correct differences among amounts of light emitted to the subject in the individual coordinates of the scanning stage.

The pulsed light emitted from the light source is transmitted to the light irradiation section through the optical waveguide. The light irradiation section irradiates the subject held by the holding section with the light through the holding section. The irradiated light is dispersed and propagated in the inside of the subject. When part of energy of the propagated light is absorbed by the light absorber, such as blood vessels, (serving as a sound source as a result), the photoacoustic waves (typically, ultrasonic waves) are generated due to thermal expansion of the light absorber. The photoacoustic waves generated in the inside of the subject are received by the probes through the holding section and the acoustic matching section. The optical waveguide and a reception section on the scanning stage perform scanning along the holding section and coordinates thereof are controlled by the stage control unit.

Light Source

In a case where the subject is a living body, pulsed light having a wavelength which may be absorbed by a specific component among components constituting the living body is emitted from the light source. The wavelength used in this embodiment is preferably a wavelength which is sufficient for propagation of light to the inside of the subject. Specifically, in the case where the subject is a living body, the wavelength is 600 nm or more and 1100 nm or less. Furthermore, to efficiently generate photoacoustic waves, a pulse width of approximately 10 to 100 nanoseconds is preferably employed. As the light source, a laser which attains large output is preferably used. However, a light emitting diode, a flash lamp, or the like may be used instead of the laser. Various lasers, such as a solid state laser, a gas laser, a dye laser, and a semiconductor laser, may be used as the laser. A timing, a waveform, intensity, and the like of the light irradiation are controlled by a light source control unit. The light source control unit may be integrated with the light source. Furthermore, the light source may be provided independently from the photoacoustic apparatus of this embodiment.

Note that the light source of this embodiment may emit light of different wavelengths.

Optical Waveguide

Examples of the optical waveguide include transmission by optical fibers, transmission by multi-joint arm using a plurality of mirrors or prisms, space transmission using a lens, a mirror, or a diffuser plate, and a combination thereof. The light from the light source may be directly incident on the optical waveguide or may be incident on the optical waveguide after a density and a form of the light are changed to an appropriate density and an appropriate form using a lens, a diffuser plate, or the like.

Light Irradiation Section

The light irradiation section is disposed on the probe supporting section so as to guide the light supplied from the optical waveguide to the subject through the probe supporting section. Material of the light irradiation section may be glass, resin, or the like, but any material may be used as long as light is transmitted through the light irradiation section. Furthermore, an antireflection film may be applied on a surface of the light irradiation section.

Light Flux Control Unit

A light flux control unit will now be described although it is not essential in the photoacoustic apparatus of this embodiment. The light flux control unit controls a direction, spread, a shape, and the like of a light flux emitted from the light irradiation section. Specifically, the light flux control unit is constituted by an optical element, such as a diffuser plate, a lens, a mirror, or the like. The light flux control unit may be disposed between the light source and the optical waveguide or between the optical waveguide and the light irradiation section.

Subject and Light Absorber

The subject and the light absorber will now be described although they are not included in the photoacoustic apparatus of this embodiment. The photoacoustic apparatus of this embodiment utilizing a photoacoustic effect mainly aims at imaging of blood vessels, diagnosis of cancers, blood vessel diseases, and the like of humans and animals, a follow-up of chemical treatment, and the like. The light absorber in the subject has a relatively high absorption coefficient although the absorption coefficient depends on a wavelength of used light. Specifically, water, fat, protein, oxygenated hemoglobin, reduced hemoglobin, or the like may be used.

Holding Section

The holding section is constituted by a member having highlight transmissivity so as to allow the light emitted to the subject to be transmitted. Furthermore, a material having acoustic impedance similar to that of the subject is preferably used so that the holding section allows the photoacoustic waves supplied from the subject to be transmitted. Examples of the holding section include polymethylpentene and a rubber sheet. Furthermore, the holding section and the subject are preferably in contact with each other through liquid, such as water, or gel so that the photoacoustic waves from the subject are efficiently received by the probes.

Acoustic Wave Detection Unit

An acoustic wave detection unit which detects photoacoustic waves generated due to pulsed light on a surface of a living body or in the inside of the living body may be reworded by probes. The probes are used to convert the photoacoustic waves into electric signals. Any probes, such as probes using a piezoelectric phenomenon, probes using light resonance, and probes using a change of an electrostatic capacitance, may be used as long as the probes are capable of detecting photoacoustic waves. Examples of the probes using the piezoelectric phenomenon include Piezo micromachined ultrasonic transducers (PMUTs) and examples of the probes using the change of the electrostatic capacitance include Capacitive micromachined ultrasonic transducers (CMUTs). The CMUT is capable of detecting photoacoustic waves in a large frequency band, and therefore, is more preferably used as the probes.

It is preferable that scanning is performed while a plurality of probes are arranged in a 2D manner or a 3D manner so that a photoacoustic image of high resolution is obtained. A reflection film, such as a gold film, may be formed on surfaces of the probes so as to return the light reflected by the surface of the subject or a surface of the holding section and the light which is scattered in the inside of subject and output from the subject to the subject.

Probe Supporting Section

The probe supporting section is used to maintain the relative positional relationship among the plurality of probes. The probe supporting section is constituted by material having high rigidity, and the material is metal, for example. A reflection film, such as a gold film, may be formed on a surface of the probe supporting section on a side near the subject so as to return the light reflected by the surface of the subject or the surface of the holding section and the light which is scattered in the inside of the subject to the subject again. The plurality of probes are preferably arranged at various angles so that the photoacoustic waves generated in the subject are received at various angles, and accordingly, the probe supporting section has a cup shape in the embodiments hereinafter. However, the probe supporting section may be a flat plate.

Acoustic Matching Section

The acoustic matching section is used to acoustically connect the holding section and the probes to each other, and the probe supporting section having the cup shape is filled with the acoustic matching section. It is preferable that the acoustic matching section allows the light from the light irradiation section to be transmitted and has an acoustic impedance similar to those of the probes. Material of the acoustic matching section may be water, gel, oil, or the like.

Position Control Unit

The position control unit controls relative positions of the light source and the subject. The position control unit of this embodiment corresponds to the stage control unit which controls the scanning stage. The scanning stage is means for causing the probe supporting section, together with the optical waveguide, to scan the holding section. The scanning stage is controlled by the stage control unit. The scanning stage is used for measurement at an arbitrary coordinate and used to cause the optical waveguide and the probe supporting section to perform scanning in a 1D manner, a 2D manner, or a 3D manner. The scanning stage may perform scanning in not only a translating direction but also a rotating direction.

Signal Processing Unit

The signal processing unit forma data associated with information on an optical characteristic value distribution, such as an absorption coefficient distribution, in the inside of the subject using the signals received by the probes. When the absorption coefficient distribution in the inside of the subject is to be calculated, in general, an initial sound pressure distribution in the inside of the subject is calculated in accordance with the signals received by the probes and light influence in the inside of the subject is taken into consideration. When the initial sound pressure distribution is formed, back projection in a time domain, for example, may be used.

Storage Unit

The storage unit is a memory including the correction table used to correct differences among amounts of light emitted to the subject in the individual stage coordinates. In the embodiments below, the stage coordinates represent coordinates in a center of the probe supporting section. The signal processing unit refers to the correction table included in the storage unit when forming the information on the inside of the subject. Note that the storage unit is not essential in the photoacoustic apparatus of this embodiment, and optical characteristic information in the inside of the subject may be formed after the signal processing unit corrects the differences among the amounts of light emitted to the subject in the individual stage coordinates.

Display Unit

The photoacoustic apparatus of this embodiment may include a display unit which displays an image formed by the signal processing unit. As the display unit, a liquid crystal display is typically used.

### Second Embodiment

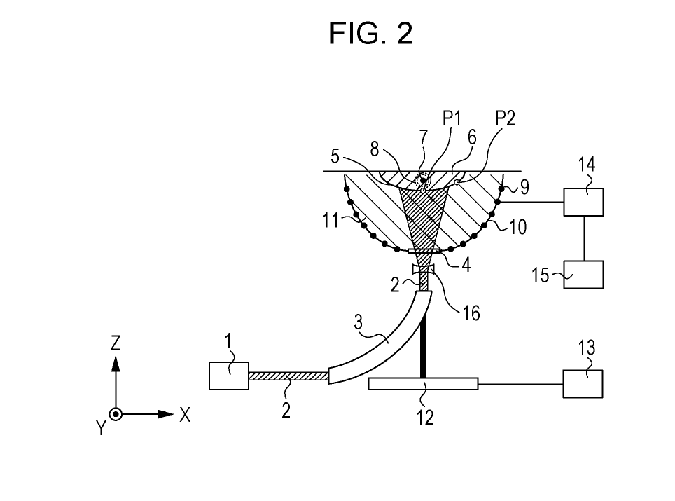

A configuration of this embodiment will be described with reference to FIG. 2. In FIG. 2, components denoted by reference numerals 1 to 15 are the same as those in FIG. 1A, and a reference numeral 16 denotes a light flux control unit. Descriptions of the same portions as the first embodiment are omitted. This is true on the third to seventh embodiments.

The light flux control unit 16 is a concave lens. A light flux of the light 2 output from the optical waveguide 3 is spread by the light flux control unit 16, transmitted through the light irradiation section 4, and incident on the subject 6 through the holding section 5. When the light is spread by the light flux control unit 15, different light attenuation amounts caused by light absorption are obtained since light path lengths of individual light beams included in the light flux in the acoustic matching section 11 are different from one another. Furthermore, since the light is spread, light densities in individual portions on which the light is incident on the subject are different from one another. Therefore, the storage unit 15 includes a correction table used to reduce differences of the light attenuation amounts in individual stage coordinates taking the light absorption and the light spread into consideration. A flow of measurement in this embodiment is the same as that illustrated in FIG. 11), and therefore, a description thereof is omitted.

As described above, since a signal processing unit forms information on the subject using the correction table generated taking the attenuation due to the spread of light into consideration even in a case where the light incident on the subject is spread, an absorption coefficient distribution may be reliably measured even if different light attenuation amounts in the acoustic matching section are obtained in different scanning positions of the light irradiation section.

### Third Embodiment

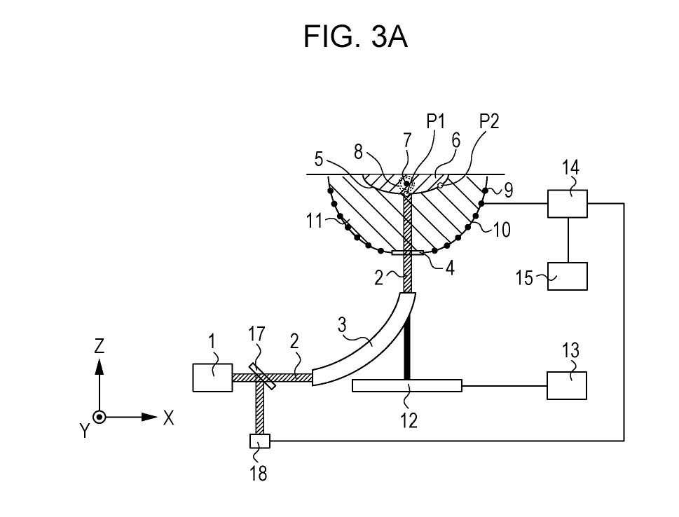

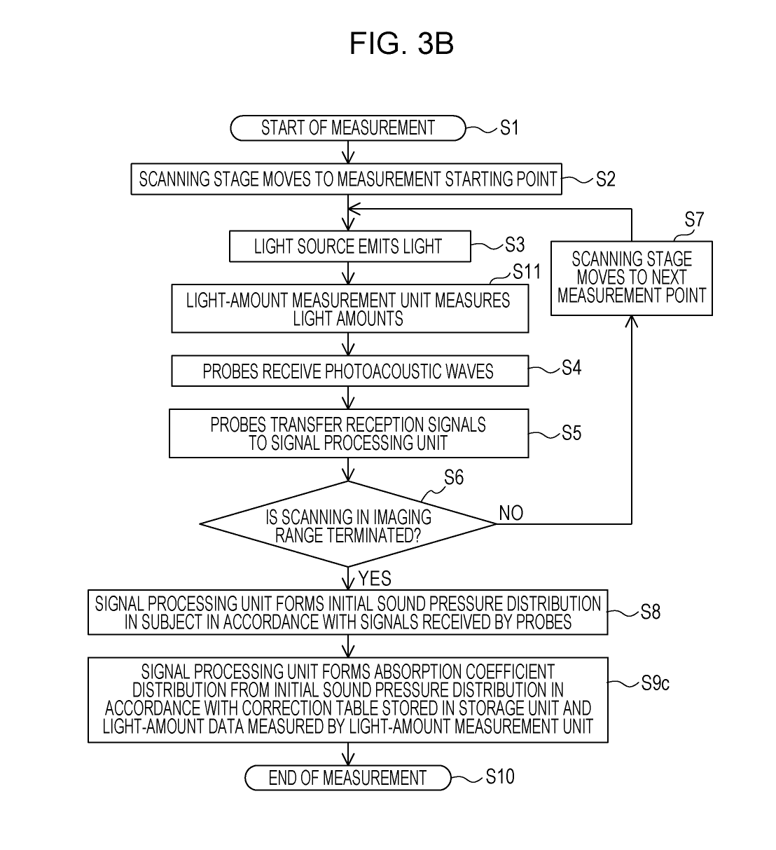

A configuration of this embodiment will be described with reference to FIG. 3. FIG. 3A is a configuration diagram of this embodiment, and FIG. 3B is a flowchart illustrating measurement of this embodiment. In FIG. 3A, components denoted by reference numerals 1 to 15 are the same as those in FIG. 1A, and a reference numeral 17 denotes a light branching unit and 18 denotes a light-amount measurement unit.

Since the components denoted by the reference numerals 1 to 15 are the same as those of the first embodiment, descriptions thereof are omitted. The light branching unit 17 is a flat glass plate having an antireflection film on a back surface thereof. The light branching unit 17 is disposed between the light source 1 and the optical waveguide 3 and reflects part of light 2 transmitted from the light source 1. The reflected light is incident on the light-amount measurement unit 18. The light-amount measurement unit 18 is a photodiode. Light amount data measured for individual pulses by the light-amount measurement unit 18 is supplied to the signal processing unit 14. The storage unit 15 stores a correction table generated taking correction of amounts of light incident on the subject 6 from the light amounts measured by the light-amount measurement unit 18 and correction of differences among amounts of light incident on the subject in individual stage coordinates into consideration. The signal processing unit 14 forms information on the inside of the subject using this correction table so that influence of the differences among the amounts of light incident on the subject in individual stage coordinates may be reduced even if different light amounts are obtained for different pulses.

Next, a flow of measurement in this embodiment is described with reference to FIG. 35. FIG. 3B is different from FIG. 1D in step S9c and step S11. When the light source 1 emits the light 2, the light-amount measurement unit 18 measures light amounts for individual pulses (S11). The signal processing unit 14 forms an absorption coefficient distribution from an initial sound pressure distribution in accordance with correction values for individual stage coordinates stored in the storage unit 15 and data on the light amounts measured for individual pulses by the light-amount measurement unit 18 (S9c).

As described above, since the light-amount measurement unit measures a portion of an amount of light emitted from the light source for each pulse in step S11 and the correction table generated taking the correction of the amounts of light incident on the subject obtained from the light amounts measured by the light-amount measurement unit and the correction of differences among the amounts of light incident on the subject in the individual stage coordinates into consideration is used in step S9c, influence of the differences of the amounts of light emitted to the subject in the individual stage coordinates may be reduced even if output of the light source varies for individual pulses. Consequently, the absorption coefficient distribution may be reliably measured even if different light attenuation amounts in the acoustic matching unit are obtained in the different individual stage coordinates.

Note that, although the signal processing unit forms the information on the inside of the subject using light amounts of pulses measured by the light-amount measurement unit in this embodiment, the information on the inside of the subject may be formed using a mean value of a plurality of pulses. Furthermore, a position of the light branching unit is not limited to a portion between the light source and the optical waveguide, and the light branching unit may be positioned in any portion as long as amounts of light incident on the subject may be estimated from the light amounts measured by the light-amount measurement unit.

### Fourth Embodiment

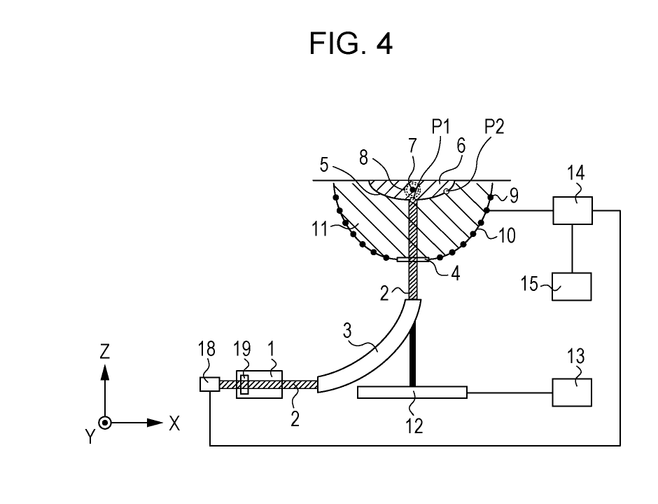

A configuration of this embodiment will be described with reference to FIG. 4. FIG. 4 is a configuration diagram of this embodiment. In FIG. 1, components denoted by reference numerals 1 to 15 and a reference numeral 18 are the same as those of the third embodiment and those in FIG. 3A, and therefore, descriptions thereof are omitted. A reference numeral 19 denotes a rear mirror.

Since the components denoted by the reference numerals 1 to 15 and the reference numeral 18 are the same as those of the third embodiment, descriptions thereof are omitted. The rear mirror 19 is one of two mirrors which have different reflectivities and which constitute a resonator of the light source 1 which is a titanium-sapphire laser. The rear mirror 19 has a higher reflectivity between the two mirrors. The rear mirror 19 has fine transmissivity, and therefore, part of the light is transmitted through the rear mirror 19 and incident on the light-amount measurement unit 18.

Since the signal processing unit 14 forms information on the inside of the subject using light amounts of individual pulses measured by the light-amount measurement unit 18, influence of differences of amounts of light incident on the subject in individual stage coordinates may be reduced even in a case where different light amounts are obtained for individual pulses. Note that a flow of measurement in this embodiment is the same as that illustrated in FIG. 3B, and therefore, a description thereof is omitted.

As described above, since the light-amount measurement unit measures a portion of an amount of light emitted from the light source for each pulse and a correction table generated taking correction of the amounts of light incident on the subject obtained from the light amounts measured by the light-amount measurement unit and correction of differences among the amounts of light incident on the subject in the individual stage coordinates into consideration is used even in the case where the rear mirror of the light source is used as a light branching unit, influence of the differences of the amounts of light incident on the subject may be reduced even if output of the light source varies for individual pulses. Consequently, the absorption coefficient distribution may be reliably measured even if different light attenuation amounts in the acoustic matching unit are obtained in different scanning positions in the light irradiation section.

### Fifth Embodiment

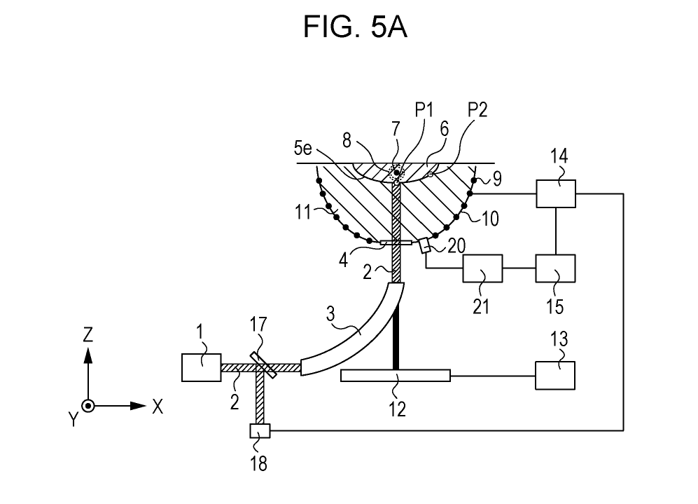

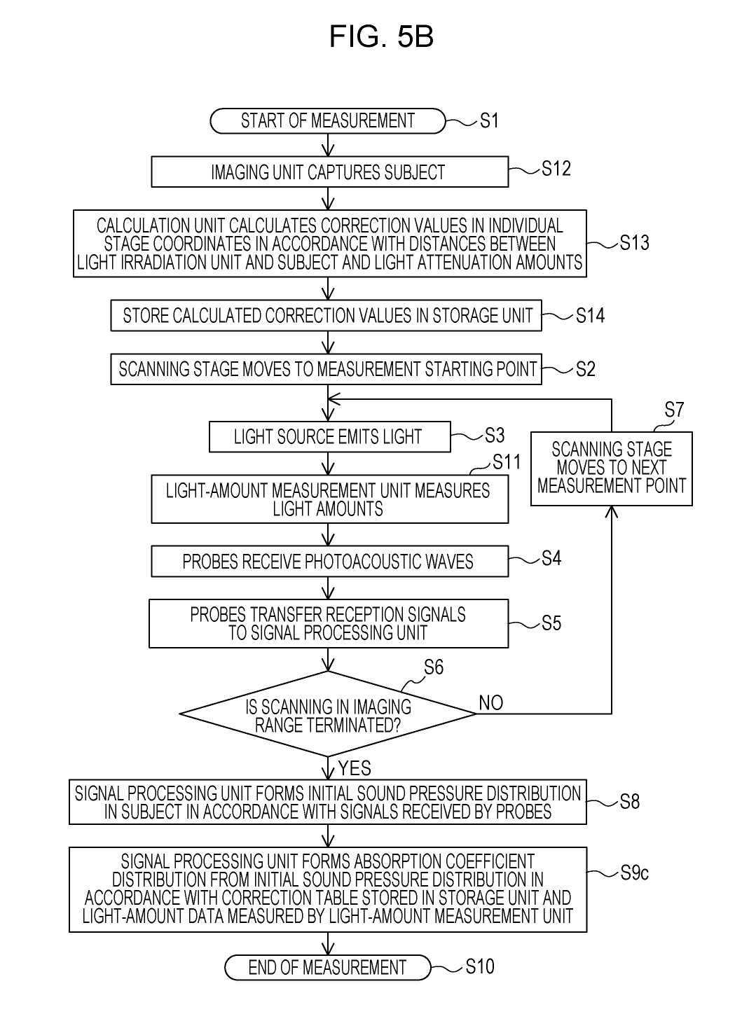

A configuration of this embodiment will be described with reference to FIG. 5. FIG. 5A is a configuration diagram of this embodiment, and FIG. 5B is a flowchart illustrating measurement of this embodiment. In FIG. 5A, components denoted by reference numerals 1 to 4 and reference numerals 6 to 18 are the same as those in FIG. 3A, and therefore, descriptions thereof are omitted. A reference numeral 5e denotes a holding section, 20 denotes an imaging unit, and 21 denotes a calculation unit.

Since the components denoted by the reference numerals 1 to 4 and the reference numerals 6 to 18 are the same as those of the third embodiment, descriptions thereof are omitted. The holding section 5e is a sheet formed of a synthetic rubber. Unlike a holding member having a cup shape which is not deformable, the rubber sheet easily stretches, and therefore, it is advantageous in that the holding section is not required to be replaced depending on a size of the subject. The imaging unit 20 captures an image of the subject 6 through the probe supporting section 10, the acoustic matching section 11, and the holding section 5e. The calculation unit 21 calculates distances between the light irradiation section 4 and the subject 6 using outputs of the imaging unit 20. Furthermore, the calculation unit 21 calculates light attenuation amounts in the acoustic matching section 11 for individual stage coordinates from the calculated distances, a light absorption coefficient of the acoustic matching section 11, and spread of light emitted to the subject 6. Furthermore, the calculation unit 21 calculates a correction table in accordance with light attenuation amounts calculated for individual stage coordinates taking correction of amounts of light emitted to the subject which are measured by the light-amount measurement unit 18 and correction of light amounts in the individual stage coordinates into consideration and stores the correction table in the storage unit 15. The signal processing unit 14 forms optical characteristic information in the inside of the subject 6 in accordance with signals received by the probes 9, light amounts measured by the light-amount measurement unit 18 for individual pulses, and the correction table stored in the storage unit 15.

Next, a flow of measurement in this embodiment is described, with reference to FIG. 5B. FIG. 5B is different from FIG. 3B only in step S12 to step S14. After an operator starts measurement, the imaging unit 20 captures an image of the subject 6 (S12). Subsequently, the calculation unit 21 calculates distances between the light irradiation section 4 and the subject 6 and calculates light attenuation amounts using the calculated distances and data on light amounts measured by the light-amount measurement unit 18. The calculation unit 21 further calculates correction values for individual stage coordinates using the calculated light attenuation amounts (S13). Thereafter, the calculation unit 21 stores the calculated correction values in the storage unit 15 (S14). Then the process proceeds to step S2.

As described above, since the calculation unit generates the correction table in accordance with a result of the image capturing performed by the imaging unit in step S12 to step S14 and the signal processing unit forms an absorption coefficient distribution in the inside of the subject in accordance with the generated correction table even in the case where the holding section is formed of a deformable member, such as a rubber sheet, the absorption coefficient distribution may be reliably measured even if different light attenuation amounts in the acoustic matching section are obtained in different scanning positions of the light irradiation section.

Note that, although the signal processing unit forms information on the inside of the subject using light amounts of the individual pulses measured by the light-amount measurement unit in this embodiment, the absorption coefficient distribution may be formed using a mean value of a plurality of pulses.

### Sixth Embodiment

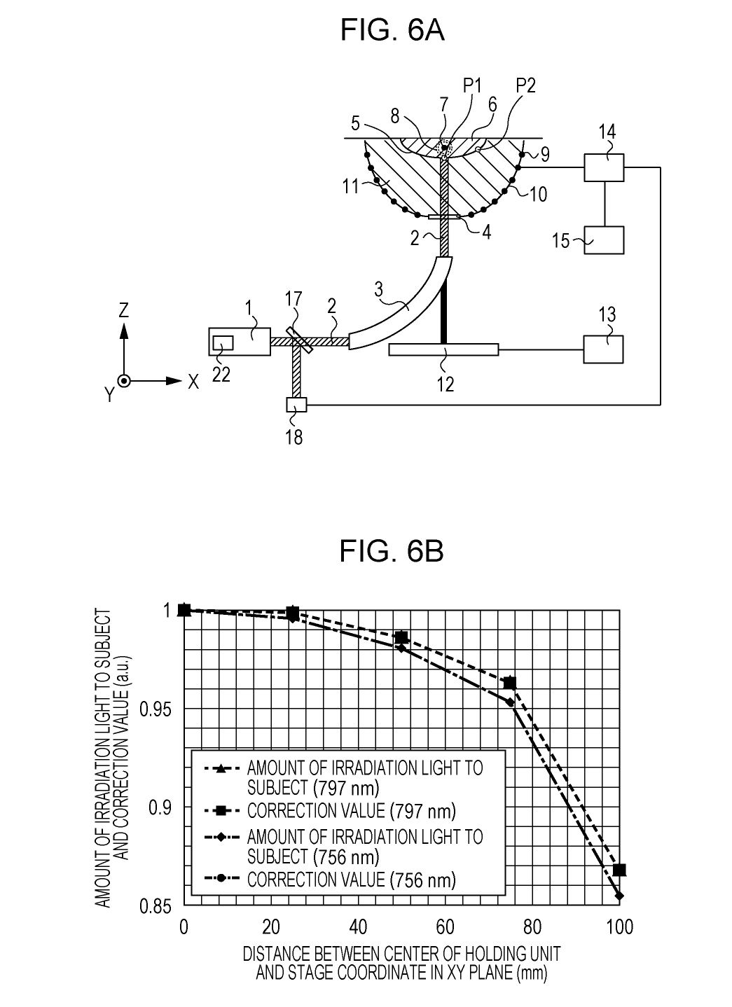

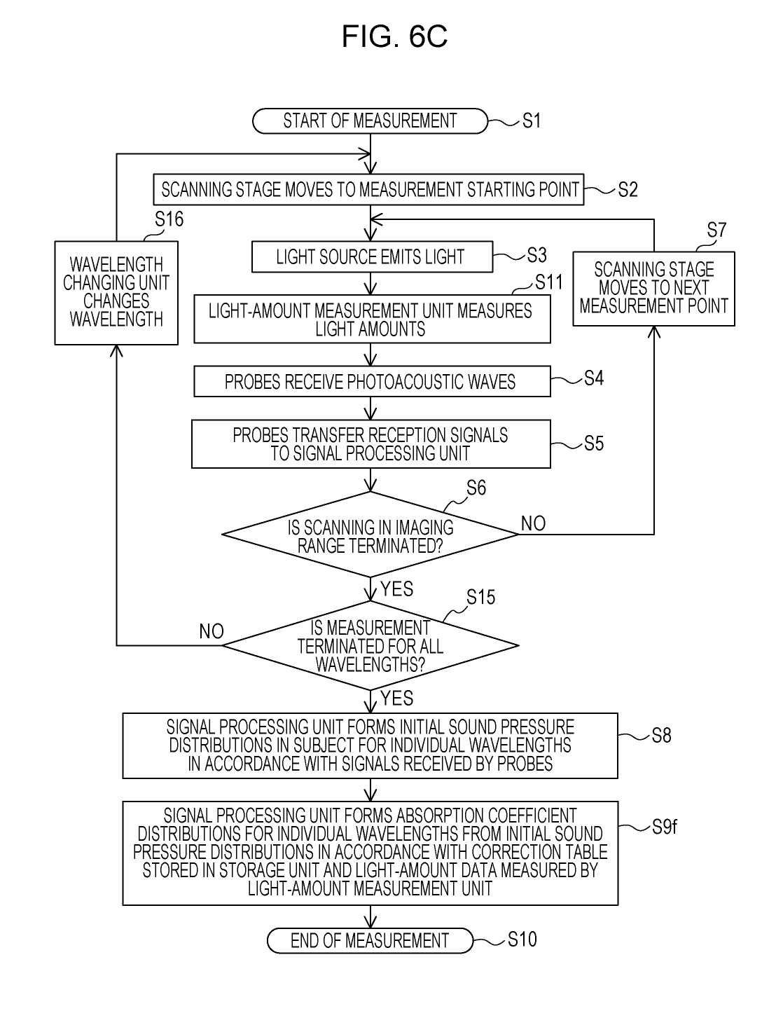

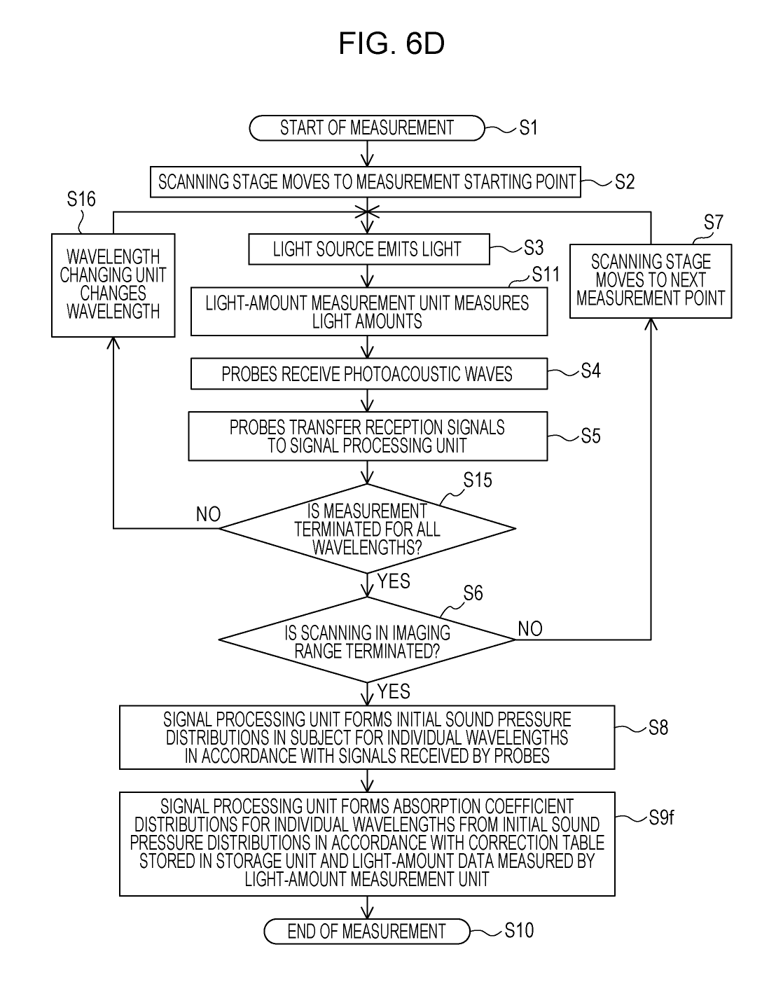

A configuration of this embodiment will be described with reference to FIG. 6. FIG. 6A is a configuration diagram of this embodiment, FIG. 6B is a diagram illustrating a correction table stored in a storage unit, and FIGS. 6C and 6D are flowcharts illustrating measurement of this embodiment. In FIG. 6A, components denoted by reference numerals 1 to 18 are the same as those in FIG. 3A, and therefore, descriptions thereof are omitted. A reference numeral 22 denotes a wavelength changing unit.

Since the components denoted by the reference numerals 1 to 18 are the same as those of the third embodiment, descriptions thereof are omitted. A light source 1 is a titanium-sapphire laser capable of emitting light of different wavelengths by operating the wavelength changing unit 22. Here, the wavelength changing unit 22 is a prism, and an oscillation wavelength of the light source 1 may be changed by changing an angle of the prism. In this embodiment, the light source 1 emits the light 2 having wavelengths of 797 nm and 756 nm.

Next, the correction table stored in the storage unit 15 and a method for using the correction table employed in the signal processing unit 14 will be described with reference to FIG. 6B. An axis of abscissa in FIG. 6B denotes a distance from a center portion P1 of the holding section 5 to a center of the probe supporting section 10 in an XY plane. When a distance from the center portion P1 of the holding section 5 to a stage coordinate in the XY plane increases, a thickness of the acoustic matching section 11 increases, and accordingly, a light attenuation amount in the acoustic matching section 11 increases. In water used as the acoustic matching section 11 in this embodiment, the light having the wavelength of 756 nm attenuates at a rate of approximately 2.5%/cm and the light having the wavelength of 797 nm attenuates at a rate of approximately 2.0%/cm. Accordingly, different amounts of light emitted to the subject 6 are obtained in different stage coordinates and also in different wavelengths. Therefore, as illustrated in FIG. 6B, as the distance from the center portion P1 of the holding section 5 to a stage coordinate in the XY plane increases, a correction value of an amount of light emitted to the subject 6 obtained from a light amount measured by the light-amount measurement unit 18 is reduced, and an appropriate value is set as a reduction rate for each wavelength so that influence of differences among amounts of light emitted to the subject is reduced. An error of an absorption coefficient distribution caused by the differences among the amounts of light emitted to the subject 6 may be reduced independently from the stage coordinates and the wavelengths.

Next, a flow of the measurement in this embodiment is described with reference to FIG. 6C. FIG. 6C is different from FIG. 1D in step S91, step S15, and step S16. When determining that imaging in a predetermined range has been terminated in step S6, a system determines whether measurement has been performed for all wavelengths (S15). When the system determines that the measurement has not been performed for at least one of the wavelengths, the wavelength changing unit selects a next wavelength (S16) and the process returns to step S2. When the system determines that the measurement has been performed for all the wavelengths, the signal processing unit 14 forms absorption coefficient distributions from initial sound pressure distributions in accordance with light amounts measured by the Might-amount measurement unit 18 (S9f). Note that, as illustrated in FIG. 6D, step S6 and step S15 may be replaced by each other. Specifically, after the measurement is performed for all the wavelengths in an arbitrary stage coordinate, the stage control unit may moves the scanning stage to a next stage coordinate.

As described above, since a wavelength of the light source is changed in step S15 and step S16 and the signal processing unit forms absorption coefficient distributions in the inside of the subject using correction values of the individual wavelengths stored in the storage unit in step S9f, influence of differences among amounts of light emitted to the subject may be reduced even if different light attenuation amounts in the acoustic matching unit disposed between the light irradiation section and the subject are obtained. Consequently, the absorption coefficient distributions may be reliably measured even if different light attenuation amounts in the acoustic matching unit are obtained in different scanning positions of the light irradiation section.

### Seventh Embodiment

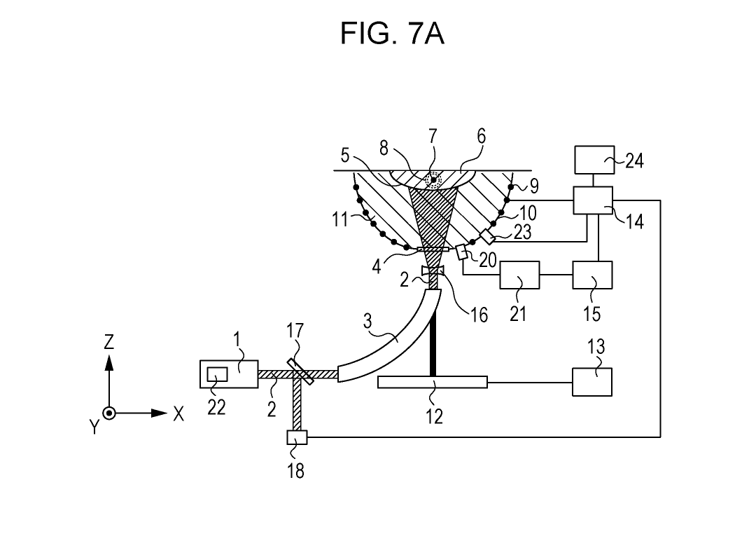

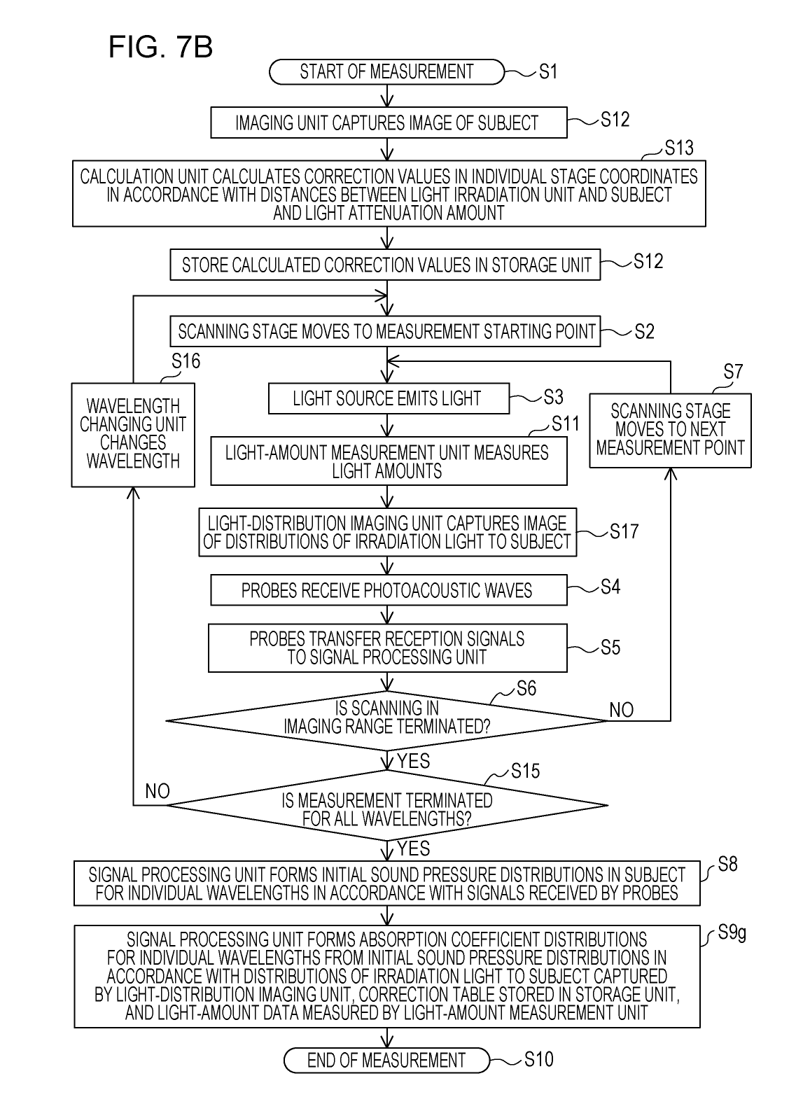

A configuration of this embodiment will be described with reference to FIG. 7. FIG. 7A is a configuration diagram of this embodiment, and FIG. 7B is a flowchart illustrating measurement of this embodiment. Components denoted by reference numerals 1 to 22 are the same as those in FIG. 5A or FIG. 6A, and therefore, descriptions thereof are omitted. A reference numeral 23 denotes a light-distribution imaging unit, and 24 denotes a display unit.

Since the components denoted by the reference numerals 1 to 22 are the same as those of the fifth embodiment or the sixth embodiment, descriptions thereof are omitted. The light-distribution imaging unit 23 captures images of distributions of light emitted to the subject 6. The light-distribution imaging unit 23 incorporates an ND filter so as to capture the light emitted to the subject 6 with appropriate intensity. The light-distribution imaging unit 23 captures images of distributions of light emitted to the subject 6 for individual stage coordinates and individual pulses and transfers data on captured light distributions to the signal processing unit 14. The signal processing unit 14 calculates light fluence in the inside of the subject 6 using the transferred light distribution data, data on light amounts for individual pulses measured by the light-amount measurement unit 18, an optical constant of the subject 6, and a correction table stored in the storage unit 15. An actually-measured value or statistical data may be used as the optical constant of the subject 6. By forming absorption coefficient distributions in the inside of the subject 6 in accordance with the calculated light fluence, light attenuation in the subject 6, influence of scattering, and variation of light amounts for individual pulses may be corrected.

Next, a flow of measurement in this embodiment is described, with reference to FIG. 7B. FIG. 7B is different from FIGS. 5B and 6C only in step S9g and step S17. In step S17, the light-distribution imaging unit 23 captures an image of a distribution of light emitted to the subject 6. After scanning and measurement in a predetermined imaging range for all wavelengths are terminated, the signal processing unit 14 forms absorption coefficient distributions from initial sound pressure distributions in accordance with correction values stored in the storage unit 15, light amounts measured by the light-amount measurement unit 18, and distributions of the irradiation light to the subject 6 captured by the light-distribution imaging unit 23.

As described above, the light-distribution imaging unit captures images of distributions of irradiation light emitted to the subject in step S17 and forms absorption coefficient distributions on the basis of the captured irradiation light distributions in step S9g, and accordingly, differences among amounts of light emitted to the subject may be reduced even if different light attenuation amounts in the acoustic matching unit disposed between the light irradiation section and the subject are obtained. Consequently, the absorption coefficient distributions may be reliably measured even if the light attenuation amounts and the irradiation light distributions in the acoustic matching unit are different for individual scanning positions of the light irradiation section and different amounts of light are obtained for individual pulses.

Note that it is not necessarily the case that the light-distribution imaging unit is disposed on the probe supporting section, and the light-distribution imaging unit may be disposed in any position as long as an image of a distribution of luminance to the subject is obtained. Furthermore, it is not necessarily the case that the light-distribution imaging unit captures an image of a distribution of irradiation light to the subject and any target may be captured as long as the distribution of the irradiation light to the subject may be estimated. Furthermore, it is not necessarily the case that the light-distribution imaging unit is incorporated in the photoacoustic apparatus of embodiment, and the light-distribution imaging unit may be externally attached.

According to the photoacoustic apparatus of the aspects of the present invention, accuracy of measurement of an absorption coefficient may be improved by obtaining information on a subject after correction is performed in accordance with amounts of light emitted to the subject.

While aspects of the present invention have been described with reference to exemplary embodiments, it is to be understood that the aspects of the invention are not limited to the disclosed exemplary embodiments. The scope of the following claims is to be accorded the broadest interpretation so as to encompass all such modifications and equivalent structures and functions.

This application claims the benefit of International Patent Application No. PCT/JP2014/084219, filed Dec. 25, 2014, which is hereby incorporated by reference herein in its entirety.

1. A photoacoustic apparatus comprising:
a photoacoustic wave detection unit configured to detect photoacoustic waves generated from a subject when a light source irradiates the subject with light and output detection signals; and
a signal processing unit configured to perform signal processing to obtain information on the subject in accordance with the detection signals,
wherein the signal processing unit obtains information on the subject after performing correction in accordance with amounts of light emitted to the subject.

2. The photoacoustic apparatus according to claim 1, wherein the signal processing unit obtains information on the subject after performing correction in accordance with differences among positions where light is emitted to the subject.

3. The photoacoustic apparatus according to claim 1 further comprising:
a storage unit including a correction table used to correct differences among amounts of light emitted to the subject that are caused by differences among positions where the light is emitted to the subject,
wherein the signal processing unit obtains information on the subject after performing correction in accordance with the amounts of light emitted to the subject.

4. The photoacoustic apparatus according to claim 1, further comprising:
an acoustic wave matching unit positioned between the light source and the subject and between the subject and the photoacoustic wave detection unit and that acoustically connects the subject and the photoacoustic wave detection unit to each other.

5. The photoacoustic apparatus according to claim 4, wherein the signal processing unit obtains information on the subject after performing correction in accordance with differences among distances in which light that is output from the light source to the subject passes the acoustic wave matching unit or light path lengths.

6. The photoacoustic apparatus according to claim 4, further comprising:
a distance measurement unit configured to measure distances in which light output from the light source to the subject passes the acoustic wave matching unit,
wherein the signal processing unit obtains information on the subject after correcting differences among amounts of light emitted to the subject caused by differences among the distances measured by the distance measurement unit.

7. The photoacoustic apparatus according to claim 4, further comprising:
an imaging unit configured to capture an image of the subject; and
a calculation unit configured calculate light attenuation amounts in the acoustic matching unit positioned between the subject and the light irradiation section for individual coordinates in a scanning stage in accordance with an output of the imaging unit,
wherein correction values used to calculate amounts of light emitted to the subject in accordance with the light attenuation amounts calculated by the calculation unit are stored in the storage unit.

8. The photoacoustic apparatus according to claim 1, further comprising:
a position control unit configured to control relative positions of the light source and the subject.

9. The photoacoustic apparatus according to claim 8, wherein information on the subject is obtained after the correction is performed in accordance with differences among the positions of the light source controlled by the position control unit.

10. The photoacoustic apparatus according to claim 1, further comprising:
a light-amount measurement unit configured to measure amounts of light output from the light source,
wherein the signal processing unit obtains information on the subject in accordance with the light amounts measured by the light-amount measurement unit.

11. The photoacoustic apparatus according to claim 10, further comprising:
a light branching unit configured to branch part of the light output from the light source,
wherein the light-amount measurement unit measures amounts of light branched by the light branching unit.

12. The photoacoustic apparatus according to claim 10, wherein
the light source is a laser having two mirrors of different reflectivities for wavelengths of emission light, and
the light-amount measurement unit measures an amount of light transmitted through one of the two mirrors having higher reflectivity.

13. The photoacoustic apparatus according to claim 1, wherein
the light source emits light of different wavelengths, and
the signal processing unit performs correction in accordance with positions where the light is emitted to the subject and the wavelengths of the light emitted from the light source.

14. The photoacoustic apparatus according to claim 1, further comprising:
a light-distribution imaging unit configured to capture images of distributions of irradiation light to the subject,
wherein the signal processing unit calculates light amount distributions inside of the subject using results of imaging performed by the light-distribution imaging unit, and obtains information on the subject in accordance with the detection signals output by the photoacoustic wave detection unit, and the light amount distributions calculated by the signal processing unit.

15. The photoacoustic apparatus according to claim 1, wherein the signal processing unit corrects amounts of light emitted to the subject.

16. The photoacoustic apparatus according to claim 1, wherein the signal processing unit corrects a parameter for calculating amounts of light emitted to the subject.

17. A photoacoustic apparatus comprising:
a photoacoustic wave detection unit configured to detect photoacoustic waves generated from a subject when a light source irradiates the subject with light and output detection signals;
a signal processing unit configured to perform signal processing to obtain information, on the subject in accordance with the detection signals; and
a storage unit configured to store a correction table including correction coefficients for irradiation positions of light emitted to the subject,
wherein the signal processing unit obtains information on the subject after correcting values of amounts of light emitted to the subject in accordance with the correction coefficients included in the correction table.
